(19) United States
(12) Patent Application Publication
(10) Pub. No.: US 2019/0388318 A1
(43) Pub. Date: Dec. 26, 2019

(54) AUTO-OXIDIZING AEROSOL FOAM HAIR COLOR

(71) Applicant: True Sons Grooming, Inc., New York, NY (US)

(72) Inventors: Anthony D'Angelo, New York, NY (US); Kevin Kramer, Grand Prairie, TX (US)

(73) Assignee: True Sons Grooming, Inc., New York, NY (US)

(21) Appl. No.: 16/448,444

(22) Filed: Jun. 21, 2019

Related U.S. Application Data
(60) Provisional application No. 62/688,069, filed on Jun. 21, 2018.

(51) Int. Cl. A61K 8/41; A61K 8/34; A61K 8/92; A61K 8/37; A61K 8/46; A61K 8/31; A61K 8/67; A61K 8/04; A61Q 5/10; A61Q 5/12

(52) U.S. Cl. CPC A61K 8/411; A61K 2800/87; A61K 8/415; A61K 8/92; A61K 8/37; A61K 8/463; A61K 8/31; A61K 8/676; A61K 8/046; A61Q 5/10; A61Q 5/12; A61K 2800/4324; A61K 2800/51; A61K 2800/522; A61K 8/347

(57) ABSTRACT

One or more techniques and/or systems are disclosed for a foam-based oxidative dye product for use on human hair. Oxidative dyes usually have complex ingredients and require a developer to initiate oxidation. A hair dye product can be produced that does not require a developer to initiate oxidation, and instead oxidizes as it is introduced to air. As an example, the hair dye product can be stored in an aerosol container, where as it exits the container it turns to foam and oxidizes. This product may result in fuller dye coverage, more control of the resulting shade, less mess, and more satisfying results for consumers.

### CROSS-RELATED APPLICATIONS

This application claims priority to U.S. Ser. No. 62/688,069, entitled FORMULA FOR AN AUTO-OXIDIZING AEROSOL FOAM HAIR COLOR UTILIZING A MINIMUM OF CARRIER BASE COMPONENTS, filed Jun. 21, 2018, which is incorporated herein by reference.

### BACKGROUND

As hair dye has evolved and become more accessible, it has become increasingly popular for people to dye their own hair from the comfort of their home. These people are rarely formally trained, and usually rely on instructions contained in the dye packaging. With oxidative permanent hair color, these instructions, amongst other things, direct people to mix the dye with a developer to obtain the expected results. The developer together with the alkaline agent, which opens up the cuticle of the hair, allows for the color to take hold. This process is the fundamental mechanism by which permanent hair color acts to produce desired results and since it requires untrained people to thoroughly mix the developer with the dye themselves it opens up multiple opportunities for mistakes, mess, and faulty hair color.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized to produce a foam-based oxidative hair-dying solution that does not utilize a developer, thus simplifying the dying process. The expanding nature of foam can allow for a larger surface area to be covered with a same or similar amount of product. A foam-based product typically provides for less mess that can occur otherwise from dripping liquid or cream based products. The foam provides cohesive forces among the bubbles of the foam to provide a relatively strong surface tension that holds them together, thus mitigating dripping. An air-oxidized foam-based product can provide ample hair dye coverage, due to the absence of lift by the developer, which allows for greater deposit of color in the hair shaft. In one implementation, the foam is formed when the dye exits an air-free container, whereupon it turns to foam via the rapid infusion of the surrounding air. In this implementation, as the dye solution infuses with air to form foam, it oxidizes, allowing the color producing reaction to proceed. This may be a slower process than oxidizing with a developer, and therefore the process can allow a more gradual control of the development of color. A permanent hair-dying product can be produced that results in less mess, ample coverage, and less risk of consumer error in application.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is an ingredient list for the hair dye formula.

FIG. 2 is a flow diagram illustrating one implementation of an example method for preparing the hair dye formula

FIG. 3 is a flow diagram illustrating one implementation of an example method for oxidation of the product and use.

### DETAILED DESCRIPTION

The claimed subject matter is now described with reference to the drawing, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

In one aspect, a foam oxidative hair dye product can be devised that does not utilize a separate developer. Further, in another aspect, methods for oxidizing the product and use of the product may be devised; and thus will be described below in this context.

An exemplary hair dye product is provided that oxidizes with air, for example, in lieu of utilizing a developer. The exemplary product may be formed through the dispersion of formula specific ingredients that, in turn, combine and form foam as they are exposed to air when they are pushed out from a pressurized, airless container. The resulting product may then be directly applied to a user's hair and used as a hair dye (e.g., permanent, semi-permanent).

The exemplary hair dye product, described herein, is different than, and improved, over current and prior hair dye products. The exemplary formula provides a simple ingredient list, does not utilize a developer to provide permanent hair dying capabilities, and can be utilized with a pressurized container. For example, current and prior products do not accomplish these results with the same level of efficiency and permanent nature.

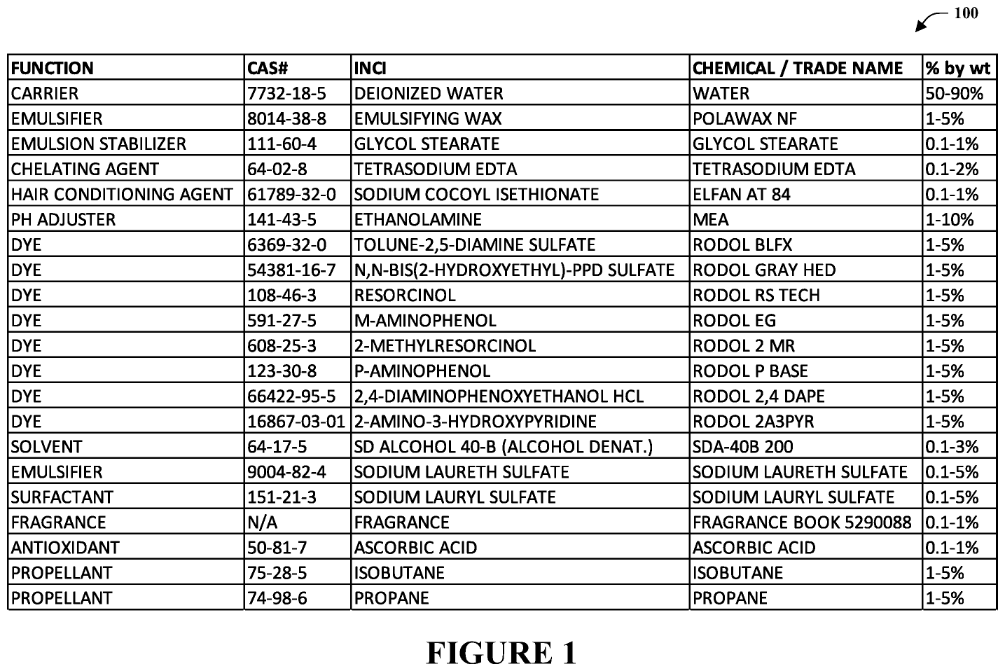

In one implementation, the exemplary hair dye product may be produced using an oxidative hair colorant or dye intermediate, a carrier, a surfactant, a chelating agent, an antioxidant, and a propellant. As one implementation, FIG. 1 illustrates a list of ingredients for an exemplary hair dye formula 100, resulting in a hair dye product for permanent hair dying capabilities.

In one implementation, the dye formula may be composed of two phases to provide foam consistency as it leaves the container. In one implementation, the two phases may comprise an oil phase and an aqueous phase. In one implementation, the oil phase may be dispersed in the aqueous phase, known as an oil in water emulsion. As illustrated in FIG. 1, the oil phase may comprise emulsifying wax and glycol stearate combined with sodium cocoyl isethionate, sodium laureth sulfate, and sodium lauryl sulfate. In this implementation, the glycol stearate and emulsifying wax may function as emulsifiers, while the sodium cocoyl isethionate, sodium lauryl sulfate and sodium laureth sulfate are surfactants that function as foaming agents, particularly when combined with a propellant. Surfactants have a special chemical structure that allows them to act as a “bridge” between two otherwise insoluble materials, in this case oil and water. Additionally, surfactants may provide detergency, emolliency, thickening and enhanced foaming, making them multi-functional.

In one implementation, the dye formula may be comprised of one or more surfactants. Surfactants may aid in the formation of desirable foam characteristics and the uniform distribution of the colorant composition on the hair surface and assist the user in rinsing the foam colorant from the hair subsequent to treatment. Surfactants may be selected for their ability to aid in the formation of an emulsion or more uniform mixture of the water, and oil or fat components of the hair colorant composition.

In one implementation, the surfactants used may be nonionic and anionic surfactants and combinations thereof. Nonionic surfactants can comprise long chain (C₁₂-C₂₂) fatty alcohols, mono, di and triglycerides and their derivatives, and alcohol ethoxylates. In one implementation, nonionic surfactants may be steareth 20, oleth 10, laureth 4, PEG-12 glyceryl dioleate, glycerol stearate, sorbitan oleate, and PPG-9 buteth-12.

In one implementation, the emulsifier or surfactant may comprise sodium cocoyl isethionate. Sodium cocoyl isethionate is the sodium salt fatty acid ester, fatty acid derived from coconut oil. In one implementation, the surfactant may comprise a self-emulsifying wax. In this implementation, the wax may be a nonionic self-emulsifying wax, for example, such as Polawax NF (Croda). In one implementation, the surfactant may be sodium lauryl sulfate or sodium laureth sulfate. In one implementation, the surfactant or emulsifier content in the total composition may be 1-10%. In one implementation, the surfactant or emulsifier content in the total composition may be 1-5%.

In one implementation, water may act as the solubilizing agent for the dye intermediates, chelating agent, anti-oxidant and alkaline agent. In this implementation, the water may also serve as a diluent for the overall composition, which may help to provide the proper rheology to the foam as it forms upon release from the container. Water also may act as a carrier to help deliver the active or functional compounds or components to the target site.

In one implementation, colorants may be formulated in the form of aqueous emulsions or mixtures. In this implementation, there may be one or more oxidizing hair colorant. The total amount and type of colorants can be dependent on a desired result. For example, a first hair dye product (e.g., for blonde hair) can comprise one or more of a first type and amount of colorants; and a second hair dye product (e.g., for black hair) can comprise a second type and amount of colorants.

In one implementation, as illustrated in FIG. 1, colorants may comprise 2,5 toluenediamine sulfate, resorcinol, m-aminophenol, p-aminophenol, n,n-bis(2-hyroxyethyl)-ppD sulfate, 2,4-diaminophenoxyethanol HCl, 2-amino-3hydroxypyridine, and 2-methyl resorcinol. The colorants are the chemicals from which the desired complete shade for the target hair is produced.

In one implementation, colorants may function as primary intermediates and can comprise one or more of: 1,4-diamino-benzene (p-phenylenediamine); 1,4-diamino-2-methyl-benzene (p-toluenediamine); 1,4-diamino-2,6-dimethyl-benzene; 1,4-diamino-3,5-diethyl-benzene; 1,4-diamino-2,5-dimethyl-benzene; 1,4-diamino-2,3-dimethylbenzene; 2-chloro-1,4-diaminobenzene; 1,4-diamino-2-(thiophen-2-yl)benzene; 1,4-diamino-2-(thiophen-3-yl)benzene; 1,4-diamino-2-(pyridin-3-yl)benzene; 2,5-diaminobiphenyl; 1,4-diamino-2-methoxymethyl-benzene; 1,4-diamino-2-aminomethylbenzene; 1,4-diamino-2-hydroxymethyl-benzene; 1,4-diamino-2-(2-hydroxyethoxy)benzene; 2-(2-(acetylamino)ethoxy)-1,4-diaminobenzene; A-phenylamino-aniline; 4-dimethylamino-aniline; 4-diethylamino-aniline; A-dipropylamino-aniline; 4-[ethyl(2-hydroxyethyl)amino]-aniline; 4-[di(2-hydroxyethyl)amino]-aniline; 4-[di(2-hydroxyethyl)amino]-2-methyl-aniline; A-[(2-methoxyethyl)amino]-aniline; 4-[(3-hydroxypropyl)amino]-aniline; 4-[(2,3-dihydroxypropyl)amino-aniline; 1,4-diamino-2-(2-hydroxyethyl)-benzene; 1,4-diamino-2-(1-methylethyl)-benzene; 1,3-bis[(4-aminophenyl)(2-hydroxyethyl)amino]-2-propanol; 1,4-bis[(4-aminophenyl)amino]-butane; 1,8-bis(2,5-diaminophenoxy)-3,6-dioxaoctane; 4-amino-phenol; 4-amino-3-methyl-phenol; 4-amino-3-(hydroxymethyl)-phenol; 4-amino-3-fluoro-phenol; A-methylamino-phenol; 4-amino-2-(aminomethyl)-phenol; 4-amino-2-(hydroxyme thy 1)-phenol; 4-amino-2-fluorophenol; 4-amino-2-[(2-hydroxyethyl)-amino] methylphenol; 4-amino-2-methyl-phenol; 4-amino-2-(methoxymethyl)-phenol; 4-amino-2-(2-hydroxyethyl)-phenol; 5-amino-salicylic acid; 2,5-diamino-pyridine; 2,4,5,6-tetraamino-pyrimidine; 4,5-diamino-1-1(2-hydroxyethyl)-1H-pyrazole; 4,5-diamino-l-(1-methylethyl)-1H-pyrazole; 4,5-diamino-1-[(4-methylphenyl)methyl]-1H-pyrazole; 1-[(4-chlorophenyl)methyl]-4,5-diamino-1H-pyrazole; 4,5-diamino-1-methyl-1H-pyrazole; 2-aminophenol; 2-amino-6-methylphenol; and 2-amino-5-methylphenol. [0032] Non-limiting examples of couplers suitable for use herein are N-(3-dimethylamino-phenyl)-urea; 2,6-diamino-pyridine; 2-amino-4-[(2-hydroxyethyl)amino]anisole; 2,4-diamino-1-fluoro-5-methylbenzene; 2,4-diamino-1-methoxy-5-methylbenzene 2,4-diamino-1-ethoxy-5-methyl-benzene; 2,4-diamino-1-(2-hydroxyethoxy)-5-methylbenzene; 2,4-di[(2-hydroxyethyl)amino]-1,5-dimethoxybenzene; 2,3-diamino-6-methoxy-pyridine; 3-amino-6-methoxy-2-(methylamino)pyridine; 2,6-diamino-3,5-dimethoxypyridine; 3,5-diamino-2,6-dimethoxypyridine; 1,3-diaminobenzene; 2,4-diamino-1-(2-hydroxyethoxy)benzene; 1,3-diamino 4-(2,3-hydroxy propoxy)benzene; 2,4-diamino-1,5-di(2-hydroxyethoxy)-benzene; 1-(2-aminoethoxy)-2,4-diaminobenzene; 2-amino-1-(2-hydroxyethoxy)-4-methylaminobenzene; 2,4-diaminophenoxyacetic acid ester; 3-[di(2-hydroxyethyl)amino]aniline; 4-amino-2-di[(2-hydroxyethyl)amino]-1-ethoxy-benzene; 5-methyl-2-(1-methyethyl)phenol; 3-[(2-hydroxyethyl)amino]aniline; 3-[(2-aminoethyl)amino]aniline; 1,3-di(2,4-diaminophenoxy)propane; di(2,4-diaminophenoxy)methane; 1,3-diamino-2,4-dimethoxybenzene; 2,6-bis(2-hydroxyethyl) amino toluene; 4-hydroxyindole; 3-dimethylaminophenol; 3-diethylaminophenol; 5-amino-2-methylphenol; 5-amino-4-fluoro-2-methyl-phenol; 5-amino-4-methoxy-2-methylphenol; 5-amino-4-ethoxy-2-methylphenol; 3-amino-2,4-dichlorophenol; 5-amino-2,4-dichlorophenol; 3-amino-2-methyl-phenol; 3-amino-2-chloro-6-methylphenol; 3-aminophenol; 2-[(3-hydroxyphenol)-amino]acetamide; 5-[(2-hydroxyethyl)amino]-4-methoxy-2-methylphenol; 5-[(2-hydroxyethyl)amino]-2-methylphenol; 3-[(2-hydroxyethyl)amino]-phenol; 3-[(2-methoxyethyl)amino]-phenol; 5-amino-2-ethyl-phenol; 5-amino-2-methoxyphenol; 2-(4-amino-2-hydroxyphenoxy)ethanol; 5-[(3-hydroxypropyl)amino]-2-methylphenol; 3-[(2,3-dihydroxypropyl)amino]-2-methylphenol; 3-[(2-hydroxyethyl)amino]-2methylphenol; 2-amino-3-hydroxypyridine; 5-amino-4-chloro-2-methylphenol; 1-naphthol; 2-methyl-1-naphthol; 1,5-dihydroxynaphthalene; 1,7-dihydroxy-naphthalene; 2,3-dihydroxynaphthalene, 2,7-dihydroxy-naphthalene; 2-methyl-1-naphthol-acetate; 1,3-dihydroxybenzene; 1-chloro-2,4-dihydroxy-benzene; 2-chloro- 1,3-dihydroxybenzene; 1,2-dichloro-2,4-dihydroxy-4-methylbenzene; 1,5-dichloro-2,4-dihydroxy-benzene; 1,3-dihydroxy-2-ethyl-benzene; 3,4-methylenedioxy-phenol; 3,4-methylenedioxy-aniline; 6-bromo-1-hydroxy-3,4-methylenedioxybenzene; 3,4-diaminobenzoic acid; 3,4-dihydroxy-6-hydroxy-1,4(2H)benzoxazine; 6-amino-3,4-dihydro-1,4(2H)-benzoxazine; 3-methyl-1-phenyl- 5-pyrazolone; 5,6-dihydroxyindole; 5,6-dihydroxyindole; 5-hydroxyindole; or 6-hydroxyindole.

In one implementation, stable salt forms of any of the colorants' molecules, described herein, may also be used.

In one implementation, various oxidation dyes may be used. Such dyes may comprise one or more of: 2-amino-4-hydroxyethylaminoanisole sulfate; 2-amino-3-hydroxypyridine; 5-amino-6-chloro-o-cresol; 4-hydroxypropylamino-3-nitrophenol; 2-methyl-5-hydroxyethyl aminophenol; 4,5 diamino-1-(2-hydroxyethyl) pyrazole sulfate; 6-amino-m-cresol; p-aminophenol cosmetic grade; m-aminophenol; o-aminophenol; 2,5 diamine toluene sulfate; 1-Naphthol; resorcinol; p-phenylenediamine; HC YELLOW 4; HC YELLOW 5; HC BLUE 2 CP; 2-nitro-p-phenylenediamine; 4-amino-3-nitrophenol; 2-chloro-p-phenylenediamine sulfate; 4-Chlororesorcinol; 2-methylresorcinol high purity; n-phenyl-p-phenylenediamine sulfate; 1-phenyl-3-methyl-5-pyrazolone; 4-amino-2-hydroxytoluene; 4-amino-m-cresol; N,N-bis-hydroxyethyl-p-phenylenediamine sulfate; p-aminophenol sulfate; m-aminophenol sulfate; p-phenylenediamine sulfate; m-phenylenediamine sulfate; 4-nitro-o-phenylenediamine; 2-amino-6-chloro-4-nitrophenol; p-methylaminophenol sulfate; 2,4diaminophenoxyethanol dihydrochloride; and any combination thereof.

In one implementation, the colorants can approximately comprise 0.1-5% of the total composition by weight. In one implementation, the colorants can comprise approximately 1-2% of the total composition by weight.

In one implementation, tetrasodium EDTA may be used as a chelating agent, and added to help with non-interference of the color formation. For example, interference can potentially occur in the presence of heavy metal ions. A chelating agent refers to a molecule or a mixture of different molecules each capable of forming a chelate with a metal ion. In one implementation, a chelating agent may comprise the salts and derivatives comprising the same functional structure as a parent chelant they are referring to, which have similar or improved chelating properties. In one implementation, a chelating agent utilized can comprise one or more of: carboxylic acids, phosphonic acids, or polyphosphoric acids, their salts and derivatives. In one implementation, a chelating agent utilized can comprise a carboxylic acid, such as aminocarboxylic acid.

In one implementation, a chelating agent can comprise one or more of the following: nitrilotriacetic acid, diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N—N′-diglutaric acid (EDDG), 2-hydroxypropylenediamine-N—N′-disuccinic acid (HPDDS), ethylenediaminetetraacetic acid (EDTA), dipicolinic acid (DPA), or salts thereof or derivatives thereof.

In one implementation, a chelating agent can comprise phosphonic acid, such as aminophosphonic acid. In one implementation, a chelating agent can comprise polyphosphoric acid, such as linear polyphosphoric acids.

In one implementation, the amount of chelating agent can comprise 0.01-1.0% of the total composition. In one implementation, the amount of chelating agent can comprise 0.1-0.3% of the total composition.

In one implementation, the addition of an alkalizing agent to the formula (e.g., 100 of FIG. 1) can provide an alkalizing effect to provide alkaline conditions that promote oxidation. In this implementation, the alkaline agent can comprise one or more of: ammonium hydroxide, alkali metal hydroxides and alkaline earth metal hydroxides; amines, such as alkanolamines, polyalkylene amines, heterocyclic amines; basic amino acids; and the like.

In one implementation, the alkaline agent can comprise one or more of: ammonium hydroxide, sodium hydroxide, potassium hydroxide, magnesium hydroxide, calcium hydroxide, urea, ethylamine, dipropyl amine, triethylamine, 1,3-diaminopropane, monoethanolamine, diethanolamine, triethanolamine, aminomethyl propanol, dimethylaminoethanol, diethylenetriamine, morpholine, diethylaminoethanol, aminoalkylpropanediol, L-arginine, lysine, oxylysine, and histidine.

In one implementation, monoethanolamine may be utilized as an alkaline agent, and as a swelling agent. For example, monoethanolamine can provide a pH of about 10 for the product to effectuate oxidation while opening, or swelling, the cortex of the hair shaft to allow product penetration.

In one implementation, the alkalizing agent may comprise 1-10% of the total composition. In one implementation, the alkalizing agent may comprise 2-6% of the total composition.

In one implementation, antioxidants can be added to the hair dye formula to mitigate oxidation before release of the product from the container. In this implementation, the antioxidant can comprise one or more of: ascorbic acid, BHT, BHA, vitamin E, erythorbic acid, and sodium sulfite. In one implementation, the antioxidant content can comprise 0.01-1% of the total composition. In one implementation, the antioxidant content can comprise 0.05-0.2% of the total composition.

In one implementation, a base formula can be devised as a standard base to which coloring agents can be added, depending on the target color and hair. In this implementation, the base formula can comprise: water, emulsifying wax and glycol stearate, sodium cocoyl isethionate, sodium laureth sulfate, sodium lauryl sulfate, colorants, tetrasodium EDTA, ethanolamine, and ascorbic acid. In this way, in this implementation, the base formula may remain consistent, while the various coloring agents are adjusted for the desired target use.

In one implementation, a carrier component can comprise water and optionally one or more cosmetically acceptable solvents or diluents. These solvents or diluents can comprise: alcohols, such as SD Alcohol 40-B, and ethers, as long as the solvents and diluents are miscible with water and do not undesirably react with other components in the composition. The use of alcohol as a diluent may help control over-growth of foam.

In one implementation, water content can comprise 50-95% of the total carrier component. In one implementation, water content can comprise 50-95% of the total composition. In one implementation, water content can comprise 80-90% of the total carrier component. In one implementation, water content can comprise 80-90% of the total composition.

In one implementation, alcohol content can comprise approximately 0.1-10.0% of the total carrier component. In one implementation, alcohol content can comprise 0.1-3.0% of the total composition. In one implementation, alcohol content can comprise 0.5-1.0% of the total carrier component. In one implementation, alcohol content can comprise 0.5-1.0% of the total composition.

In one implementation, other cosmetically suitable ingredients, including but not limited to fragrance, humectants, and emollients may be added to the hair dye formula, resulting in the hair dye product.

In one implementation, hydrocarbon based propellants may be added to the composition, and used as a propellant to force the product out of the consider under pressure, during use. In one implementation, the propellant can comprise one or more of: propane, isobutane, n-butane, or mixtures thereof, or dimethyl ether. In one implementation, the propellant can comprise A-46, which is a mixture of propane and isobutane. In one implementation, a propellant is added that can comprise 1-30% of the weight of the total composition. In one implementation, a propellant is added that can comprise 2-15% of the weight of the total composition. In one implementation, a propellant is added that can comprise 3-5% of the weight of the total composition.

In one implementation, the base composition of the exemplary hair dye product may comprise an oxidative hair colorant and/or dye intermediate, a carrier, a surfactant, a chelating agent, an antioxidant, and a propellant. In one implementation, the resulting hair dye product can be contained in an air free container. In one implementation, the air free container can comprise an aerosol-type canister, along with related foam dispensing components, such as a valve and actuator.

In one aspect, the method for making the hair dye product, described herein, can comprise making the base composition, and adding hair dying agents as needed for respective target uses. In one implementation, the base composition may be made in a tank, such as a suitable jacketed, stainless steel tank. For example, the tank may be heated with steam and/or cooled with chilled water. As one example, the tank may utilize one or more mixers, such as one or more of: an impeller style mixer, a homogenizing mixer, and a sweep mixer. In one implementation, the respective mixers can be controlled to allow variable speeds for different levels of mixing or agitation.

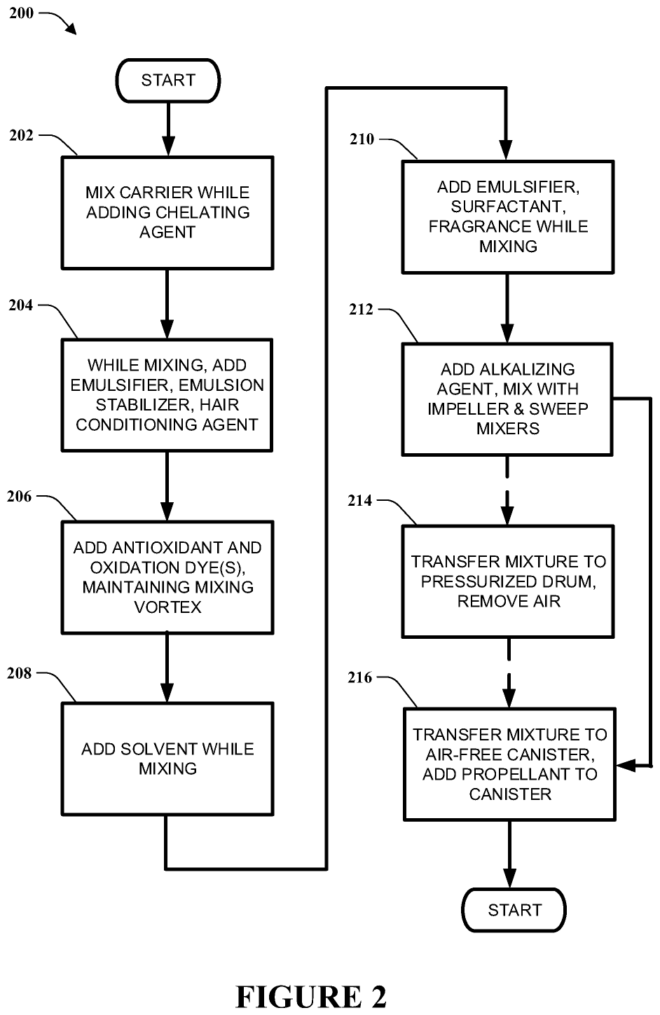

FIG. 2 is a flow diagram illustrating one implementation of an example method 200 for making a hair dye product described herein. In the example method 200, at 202, the carrier is added to a main tank, and the chelating agent can be added while mixing slowly until distributed in the mixture, visually appearing uniform in color and consistency and while heating to about 70-80 degrees C. (Phase A). At 204, the emulsifier, emulsion stabilizer, and hair conditioning agent may be added to the main tank while mixing, at least until substantially dissolved, which is indicated by no visible lumps or particles of the added ingredient, appearing homogeneous, and while cooling to about 40-50 degrees C. (Phase B).

At 206, the antioxidant and oxidation dyes can be added one-by-one, for example, the addition of the antioxidant may be followed by the addition of the oxidation dyes to the main tank, while mixing slowly, at least until distributed in the mixture, which is indicated by visually appearing uniform in color and consistency. Further, in one implementation, the mixing speed in the main tank can be adjusted, if necessary, to maintain a vortex (Phase C). At 208, the solvent may be added to the main tank while mixing slowly until distributed in the mixture visually appearing uniform in color and consistency and while cooling to about 35 degrees C. (Phase D).

At 210, of FIG. 2, the emulsifier, surfactant, and fragrance may be added one-by-one, for example, in the order of emulsifier, followed by the surfactant and then the fragrance, to the main tank while mixing slowly, at least until the ingredients are distributed in the mixture, indicated by visually appearing uniform in color and consistency. Further, in one implementation, the mixing speed can be adjusted to mitigate the creation of foam (Phase E). At 212, the alkalizing agent may be added to the mixture when the temperature has reached approximately 25 degrees C. In one implementation, the mixture can be mixed with the impeller and sweep mixers for approximately 5 to 10 minutes.

In one optional implementation, at 214, the resulting mixture (e.g., the base composition) can be pumped into a drum that is pressurized to approximately between 5-15 PSI, and preferably 5-10 PSI. In one implementation, the drum can be placed under a vacuum of approximately 5-10 mmHg, in order to substantially remove ambient air (Phase F). At 216, the base composition may be filled into an air free container followed by propellant, for example, from the drum at 214, or from the mixing container at 212. As one example, volumetric cylinders with pistons can deliver a set amount of product to the respective canisters. In this example, the stroke of the cylinders may be adjusted to obtain a desired amount (e.g., appropriate for the target use) of propellant and formula. In one implementation, the propellant may be introduced by a “through the valve method.” The formula (e.g., bulk concentrate) may be placed into the canister (e.g., aerosol can), and a valve may be fitted onto the canister. Subsequently, a vacuum of approximately 5-22 mmHg (e.g., to help remove air) can be applied to the can, and the valve can be engaged (e.g., crimped) onto the canister to seal it. In this implementation, the propellant can be injected into the canister through the valve.

In one implementation, the propellant is introduced by an “under the cup method.” In this implementation, the injection of the propellant, vacuuming and crimping may be done all together (Phase G above).

In one implementation, dependent upon the target user's hair, and intended use, the oxidative dyes may be added to the base formula, as needed. For example, the dyes can be added in the order of Resorcinol, Toluene-2,5-diamine sulfate, m-Aminophenol, p-Aminophenol, 2-Amino-3-hydroxypyridine, N,N-Bis(2-hydroxyethyl)-ppD sulfate, 2,4-Diaminophenoxyethanol HCl, and 2-Methylresorcinol, excluding any oxidative dye(s) that may not be needed for the color formula.

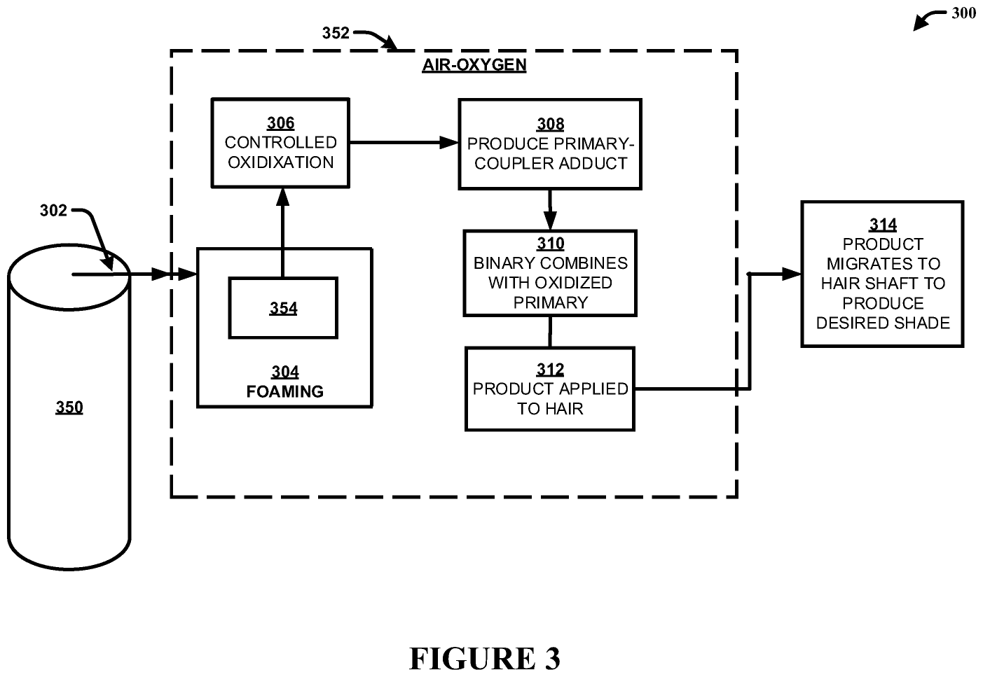

FIG. 3 is a flow diagram illustrating one implementation of an example method 300 for using one or more of the hair dye products described herein. In FIG. 3, in one implementation, the exemplary hair dye product may be contained in a container 350 (e.g., an aerosol container). The container 350 may be sealed closed thereby keeping the exemplary hair dye product in a substantially oxygen free environment and mitigating premature oxidation prior to dispensing of the product. In this implementation, oxidation can occur when the product is released, at 302, from the container 350. For example, the product can be pushed out of the canister 300 by opening the valve, such as by using an actuator, which results in the pressurized propellant forcing the product out through the valve. The product is released into the environment 352, at 302, which includes oxygen and other typical atmospheric gases. Upon release, at 304, the product forms a foam with the rapid infusion of the surrounding air.

In this implementation, at 306, the exemplary hair dye product may be oxidized by the air as it exits an air free container 350, such as an aerosol container or bag-on-valve delivery system. Control of the oxidation begins at 306, which is “built in” due to the concentrations of dye intermediates 354, primaries and couplers, combining with each other as they consume the fixed amount of oxygen in the air at a constant rate.

At 308, a primary-coupler adduct 356 is produced when the overall reaction between a given primary and coupler begins with a molecule of primary first reacting with oxygen, which converts it to an “oxidized” intermediate ready to combine with a molecule of coupler transferring its energy to the primary-coupler adduct produced. In this implementation, this adduct is known as a binary. At 310, the binary in turn combines with another “oxidized” primary, which in turn combines with another coupler molecule to continue or propagate the color-producing reaction sequence (335).

Thus, a series of short chained color producing polymeric segments is produced. At 314, while the segments are forming, they are migrating into the hair shaft through the cortex which has been “swelled” by an alkaline agent. For example, in a given product, there are a number of different primaries and couplers reacting simultaneously in this manner to form a complete shade. In this example, the number of these reactions which occur may be directly proportional to the concentration of the various primaries and couplers in the formula, to provide the desired shade of hair color. For example, the greater the number of reactions that may occur in a set development time, the deeper the resulting hair color may result. Therefore, the product, together with the oxygen in the air may control how the dye will be oxidized. The air free container may also deter the premature oxidation of the dye product prior to application.

In one implementation, the exemplary hair dye product may be applied weekly. In this implementation, due to the slow nature of oxidation the hair dye product may be used repeatedly over a short period of time to maintain the desired level of coverage.

In one implementation, a process for application of the exemplary hair dye product may consist in shaking the air free container, in order to activate the foam. Then dispense foam, in one embodiment by holding the air free container at a 45-degree angle pointing actuator into the palm of the user's hand, and the user may wear gloves. In one embodiment, the user may dispense golf ball sized portions. The exemplary hair dye product may be applied to hair, and may massage the product through the hair with fingers or comb, and may wait 10 to 30 minutes, and preferably 15 to 20 minutes. After the elapsed time, the user may thoroughly wash the product out, preferably with water. The user may wash the product out at least until the water runs clear. In one example, the result of this method of application can be a desired hair shade of the user's hair.

Moreover, the word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

Furthermore, the claimed subject matter may be implemented as a method, apparatus or article of manufacture using standard engineering techniques to produce the disclosed subject matter. Of course, those skilled in the art will recognize many modifications may be made to this configuration without departing from the scope or spirit of the claimed subject matter. The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. An oxidizing hair dye product, comprising:
a solution comprising:
a colorant;
a carrier;
a surfactant;
an antioxidant;
a chelating agent;
an alkalizing agent.

2. The hair dye product of claim 1, wherein the colorant(s) comprise 0.5-5% of the total composition by weight and are selected from a group consisting of oxidative hair dye(s): 2,5 toluenediamine sulfate, resorcinol, m-aminophenol, p-aminophenol, n,n-bis(2-hydroxyethyl)-ppD sulfate, 2,4-diaminophenoxyethanol HCl, 2-amino-3 hydroxypyridine, 2-methyl resorcinol, 1,4-diamino-benzene (p-phenylenediamine); 1,4-diamino-2-methyl-benzene (p-toluenediamine); 1,4-diamino-2,6-dimethyl-benzene; 1,4-diamino-3,5-diethyl-benzene; 1,4-diamino-2,5-dimethyl-benzene; 1,4-diamino-2,3-dimethylbenzene; 2-chloro-1,4-diaminobenzene; 1,4-diamino-2-(thiophen-2-yl)benzene; 1,4-diamino-2-(thiophen-3-yl)benzene; 1,4-diamino-2-(pyridin-3-yl)benzene; 2,5-diaminobiphenyl; 1,4-diamino-2-methoxymethyl-benzene; 1,4-diamino-2-aminomethylbenzene; 1,4-diamino-2-hydroxymethyl-benzene; 1,4-diamino-2-(2-hydroxyethoxy)benzene; 2-(2-(acetylamino)ethoxy)-1,4-diaminobenzene; A-phenylamino-aniline; 4-dimethylamino-aniline; 4-diethylamino-aniline; A-dipropylamino-aniline; 4-[ethyl(2-hydroxyethyl)amino]-aniline; 4-[di(2-hydroxyethyl)amino]-aniline; 4-[di(2-hydroxyethyl)amino]-2-methyl-aniline; A-[(2-methoxyethyl)amino]-aniline; 4-[(3-hydroxypropyl)amino]-aniline; 4-[(2,3-dihydroxypropyl)amino-aniline; 1,4-diamino-2-(2-hydroxyethyl)-benzene; 1,4-diamino-2-(1-methylethyl)-benzene; 1,3-bis[(4-aminophenyl)(2-hydroxyethyl)amino]-2-propanol; 1,4-bis[(4-aminophenyl)amino]-butane; 1,8-bis(2,5-diaminophenoxy)-3,6-dioxaoctane; 4-amino-phenol; 4-amino-3-methyl-phenol; 4-amino-3-(hydroxymethyl)-phenol; 4-amino-3-fluoro-phenol; A-methylamino-phenol; 4-amino-2-(aminomethyl)-phenol; 4-amino-2-(hydroxyme thy 1)-phenol; 4-amino-2-fluorophenol; 4-amino-2-[(2-hydroxyethyl)-amino] methylphenol; 4-amino-2-methyl-phenol; 4-amino-2-(methoxymethyl)-phenol; 4-amino-2-(2-hydroxyethyl)-phenol; 5-amino-salicylic acid; 2,5-diamino-pyridine; 2,4,5,6-tetraamino-pyrimidine; 4,5-diamino-1-1(2-hydroxyethyl)-1H-pyrazole; 4,5-diamino-l-(1-methylethyl)-1H-pyrazole; 4,5-diamino-1-[(4-methylphenyl)methyl]-1H-pyrazole; 1-[(4-chlorophenyl)methyl]-4,5-diamino-1H-pyrazole; 4,5-diamino-1-methyl-1H-pyrazole; 2-aminophenol; 2-amino-6-methylphenol; and 2-amino-5-methylphenol. [0032] Non-limiting examples of couplers suitable for use herein are N-(3-dimethylamino-phenyl)-urea; 2,6-diamino-pyridine; 2-amino-4-[(2-hydroxyethyl)amino]anisole; 2,4-diamino-1-fluoro-5-methylbenzene; 2,4-diamino-1-methoxy-5-methylbenzene 2,4-diamino-1-ethoxy-5-methyl-benzene; 2,4-diamino-1-(2-hydroxyethoxy)-5-methylbenzene; 2,4-di[(2-hydroxyethyl)amino]-1,5-dimethoxybenzene; 2,3-diamino-6-methoxy-pyridine; 3-amino-6-methoxy-2-(methylamino)pyridine; 2,6-diamino-3,5-dimethoxypyridine; 3,5-diamino-2,6-dimethoxypyridine; 1,3-diaminobenzene; 2,4-diamino-1-(2-hydroxyethoxy)benzene; 1,3-diamino 4-(2,3-hydroxypropoxy)benzene; 2,4-diamino-1,5-di(2-hydroxyethoxy)-benzene; 1-(2-aminoethoxy)-2,4-diaminobenzene; 2-amino-1-(2-hydroxyethoxy)-4-methylaminobenzene; 2,4-diaminophenoxyacetic acid ester; 3-[di(2-hydroxyethyl)amino]aniline; 4-amino-2-di[(2-hydroxyethyl)amino]-1-ethoxy-benzene; 5-methyl-2-(1-methyethyl)phenol; 3-[(2-hydroxyethyl)amino]aniline; 3-[(2-aminoethyl)amino]aniline; 1,3-di(2,4-diaminophenoxy)propane; di(2,4-diaminophenoxy)methane; 1,3-diamino-2,4-dimethoxybenzene; 2,6-bis(2-hydroxyethyl) amino toluene; 4-hydroxyindole; 3-dimethylaminophenol; 3-diethylaminophenol; 5-amino-2-methylphenol; 5-amino-4-fluoro-2-methyl-phenol; 5-amino-4-methoxy-2-methylphenol; 5-amino-4-ethoxy-2-methylphenol; 3-amino-2,4-dichlorophenol; 5-amino-2,4-dichlorophenol; 3-amino-2-methyl-phenol; 3-amino-2-chloro-6-methylphenol; 3-aminophenol; 2-[(3-hydroxyphenol)-amino]acetamide; 5-[(2-hydroxyethyl)amino]-4-methoxy-2-methylphenol; 5-[(2-hydroxyethyl)amino]-2-methylphenol; 3-[(2-hydroxyethyl)amino]-phenol; 3-[(2-methoxyethyl)amino]-phenol; 5-amino-2-ethyl-phenol; 5-amino-2-methoxyphenol; 2-(4-amino-2-hydroxyphenoxy)ethanol; 5-[(3-hydroxypropyl)amino]-2-methylphenol; 3-[(2,3-dihydroxypropyl)amino]-2-methylphenol; 3-[(2-hydroxyethyl)amino]-2methylphenol; 2-amino-3-hydroxypyridine; 5-amino-4-chloro-2-methylphenol; 1-naphthol; 2-methyl-1-naphthol; 1,5-dihydroxynaphthalene; 1,7-dihydroxy-naphthalene; 2,3-dihydroxynaphthalene, 2,7-dihydroxy-naphthalene; 2-methyl-1-naphthol-acetate; 1,3-dihydroxybenzene; 1-chloro-2,4-dihydroxy-benzene; 2-chloro-1,3-dihydroxybenzene; 1,2-dichloro-2,4-dihydroxy-4-methylbenzene; 1,5-dichloro-2,4-dihydroxy-benzene; 1,3-dihydroxy-2-ethyl-benzene; 3,4-methylenedioxy-phenol; 3,4-methylenedioxy-aniline; 6-bromo-1-hydroxy-3,4-methylenedioxybenzene; 3,4-diaminobenzoic acid; 3,4-dihydroxy-6-hydroxy-1,4(2H)benzoxazine; 6-amino-3,4-dihydro-1,4(2H)-benzoxazine; 3-methyl-1-phenyl-5-pyrazolone; 5,6-dihydroxyindole; 5,6-dihydroxyindole; 5-hydroxyindole; 6-hydroxyindole, and the salt forms of any colorant molecules that form stable salts.

3. The hair dye product of claim 2, wherein the oxidative hair dye(s) are selected from a group consisting of: 2-amino-4-hydroxyethylaminoanisole sulfate; 2-amino-3-hydroxypyridine; 5-amino-6-chloro-o-cresol; 4-hydroxypropylamino-3-nitrophenol; 2-methyl-5-hydroxyethyl aminophenol; 4,5 diamino-1-(2-hydroxyethyl) pyrazole sulfate; 6-amino-m-cresol; p-aminophenol cosmetic grade; m-aminophenol; o-aminophenol; 2,5 diamine toluene sulfate; 1-Naphthol; resorcinol; p-phenylenediamine; HC YELLOW 4; HC YELLOW 5; HC BLUE 2 CP; 2-nitro-p-phenylenediamine; 4-amino-3-nitrophenol; 2-chloro-p-phenylenediamine sulfate; 4-Chlororesorcinol; 2-methylresorcinol high purity; n-phenyl-p-phenylenediamine sulfate; 1-phenyl-3-methyl-5-pyrazolone; 4-amino-2-hydroxytoluene; 4-amino-m-cresol; N,N-bis-hydroxyethyl-p-phenylenediamine sulfate; p-aminophenol sulfate; m-aminophenol sulfate; p-phenylenediamine sulfate; m-phenylenediamine sulfate; 4-nitro-o-phenylenediamine; 2-amino-6-chloro-4-nitrophenol; p-methylaminophenol sulfate; 2,4diaminophenoxyethanol dihydrochloride; and any combination thereof.

4. The hair dye product of claim 1, wherein the carrier(s) are comprised of water and a cosmetically acceptable solvents or diluents;
wherein the cosmetically acceptable solvents or diluents are alcohols, such as SD Alcohol 40-B, and ethers, that are miscible with water and do not undesirably react with other components in the composition;
wherein the water comprises 50-95% of the total composition by weight;
wherein the alcohol comprises 0.1-3.0% of the total composition by weight.

5. The hair dye product of claim 1, wherein the surfactant(s) comprise 1-10% of the total composition by weight and are selected from a group consisting of sodium cocoyl isethionate, sodium laureth sulfate, and sodium lauryl sulfate, glycol stearate, emulsifying wax, long chain (C12-C22) fatty alcohols, mono, di and triglycerides and their derivatives, alcohol ethoxylates, steareth 20, oleth 10, laureth 4, PEG-12 glyceryl dioleate, glycerol stearate, sorbitan oleate, PPG-9 buteth-12, and nonionic self-emulsifying wax.

6. The hair dye product of claim 1, wherein the antioxidant(s) comprise 0.01-1% of the total composition by weight and are selected from a group consisting of ascorbic acid, BHT, BHA, vitamin E, erythorbic acid, or sodium sulfite.

7. The hair dye product of claim 1, wherein the chelating agent(s) comprise 0.01-1.0% of the total composition by weight and are selected from a group consisting of tetrasodium EDTA, carboxylic acids, phosphonic acids, polyphosphoric acids, nitrilotriacetic acid, diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N—N′-diglutaric acid (EDDG), 2-hydroxypropylenediamine-N—N′-disuccinic acid (HPDDS), ethylenediaminetetraacetic acid (EDTA), dipicolinic acid (DPA), including all salts and derivatives comprising the same functional structure as the parent chelant.

8. The hair dye product of claim 1, wherein the alkalizing agent(s) comprise 1-10% of the total composition by weight and are selected from a group consisting of ammonium hydroxide, alkali metal hydroxides, alkaline earth metal hydroxides; amines, alkanolamines, polyalkylene amines, heterocyclic amines; basic amino acids, sodium hydroxide, potassium hydroxide, magnesium hydroxide, calcium hydroxide, urea, ethylamine, dipropyl amine, triethylamine, 1,3-diaminopropane, monoethanolamine, diethanolamine, triethanolamine, aminomethyl propanol, dimethylaminoethanol, diethylenetriamine, morpholine, diethylaminoethanol, aminoalkylpropanediol, L-arginine, lysine, oxylysine, histidine, and monoethanolamine.

9. A method of using an oxidizing hair dye, comprising:
using an air free pressurized container to apply a hair dye product, the air free pressurized container comprising:
a propellant to provide pressure to release the product from the container;
an actuator comprising a valve to let the product out of the canister upon activation of the actuator; and
the hair dye product comprising:
a colorant;
a carrier;
a surfactant;
an antioxidant;
a chelating agent;
an alkalizing agent;
wherein the using comprises a user activating the actuator to release the product from the container using the propellant, wherein the release of the product from the canister into the atmosphere comprising oxygen initiates an oxidizing process in the product.

10. The method of oxidizing hair dye of claim 9, wherein releasing the product from the air free pressurized container comprises releasing the product from an aerosol container.

11. The method of oxidizing hair dye of claim 9, wherein the propellant(s) comprise 1-30% of the total composition by weight and are selected from a group consisting of hydrocarbon, propane, isobutane, n-butane, or mixtures thereof, dimethyl ether, A-46, a mixture of propane and isobutane.

12. The method of claim 9, comprising:
dispensing the product by activating the actuator, resulting in oxidization of the product;
apply the oxidized product to a user's hair;
waiting for a shade development time;
washing the product out of the user's hair.

13. A method of preparing a hair dye product:
mixing a carrier, a chelating agent, a first emulsifier, an emulsion stabilizer, a hair conditioning agent, an antioxidant, a second emulsifier, a colorant, a solvent, a surfactant, a fragrance, and an alkalizing agent together; and
disposing the mixture into an aerosol container with a propellant.

14. The method of preparing a hair dye product of claim 12, wherein the mixing comprises first mixing the carrier in a tank and then mixing in the chelating agent while heating the tank to 70-80 degrees C.

15. The method of preparing a hair dye product of claim 123, wherein the mixing comprises adding and mixing the first emulsifier, emulsion stabilizer, and hair conditioning agent to the tank while cooling to 40-50 degrees.

16. The method of preparing a hair dye product of claim 14, wherein the mixing comprises adding the antioxidant to the tank and mixing, then adding by the colorant to the tank and mixing while maintaining a mixing vortex.

17. The method of preparing a hair dye product of claim 15, wherein the mixing comprises adding the solvent to the tank and mixing at a temperature of 30-40 degree C.

18. The method of preparing a hair dye product of claim 16, wherein the mixing comprises adding the second emulsifier, surfactant, and fragrance to the tank one-by-one and mixing while mitigating the creation of foam.

19. The method of preparing a hair dye product of claim 17, wherein the mixing comprises cooling the tank to 20-30 degrees C. and mixing for 5-10 minutes, then adding the alkalizing agent to the tank.

20. The method of preparing a hair dye product of claim 18, comprising one or more of:
disposing the mixture into a drum that is pressurized to 15 PSI, then vacuuming the drum to 5-10 mmHg; and
canning the mixture by filling an air free container, and adding a propellant.
